(19) United States
(12) Patent Application Publication
(10) Pub. No.: US 2006/0235090 A1
(43) Pub. Date: Oct. 19, 2006

(54) Process for synthesis of methanol

(75) Inventors: Poul E. Nielsen, Fredensborg (DK); Susanne L. Jorgensen, Holte (DK)

Correspondence Address: DICKSTEIN SHAPIRO LLP, 1825 EYE STREET NW, Washington, DC, 20006-5403, US

(73) Assignee: HALDOR TOPSOE A/S, Kgs. Lyngby (DK)

(21) Appl. No.: 10/541,224

(22) PCT Filed: Dec. 9, 2003
(86) PCT No.: PCT/EP03/13942

§ 371 Date: Apr. 10, 2006

(30) Foreign Application Priority Data
Dec. 21, 2002 (DK) PA 2002 02000

(51) Int. Cl. C07C 27/26

(52) U.S. Cl. 518/726

(57) ABSTRACT

The invention provides a process for production of methanol from a feed stream rich in hydrogen carbon monoxide and carbon dioxide. The feed stream is converted to a converted process stream comprising methanol and small amounts of higher alcohols, aldehydes and ketones in the presence of a catalyst active in conversion of hydrogen and carbon monoxide into methanol. The converted process stream is cooled to a cooled process stream to 20-200° C. The cooled process stream is hydrogenated into a hydrogenated process stream rich in methanol and depleted in aldehydes and ketones in presence of a hydrogenation catalyst. The catalyst is active in conversion of aldehydes and ketones into alcohols in a process stream rich in methanol and further comprising hydrogen, carbon monoxide and carbon dioxide. The hydrogenated process stream is cooled to a cooled, condensed process stream, and subsequently the cooled, condensed process stream is separated into a gas phase and a liquid crude methanol phase.

### BACKGROUND OF THE INVENTION

1. Field of the Invention

The invention relates to an improved process for production of methanol and in particular chemical grade methanol from hydrogen, carbon monoxide and carbon dioxide.

2. Description of Related Art

Methanol is a widely used product and intermediate product as well. It is industrially produced by different catalytic processes.

It is known from U.S. Pat. No. 5,243,095 that alcohols can be produced by hydrogenation of the feed materials aldehydes and ketones. With these raw materials, hydrogenation takes place over a catalyst containing Cu, Fe, Al and/or Mn at 250-350° C.

Similarly, U.S. Pat. No. 3,925,490 describes hydrogenation of aldehydes and ketones, which are the desired intermediaries products in the traditional oxo process for production of alcohols. The hydrogenation takes place over a Cu, Cr catalyst at 100-200° C.

A conversion of hydrogen and carbon monoxide rich synthesis gas to methanol is described in U.S. Pat. No. 4,540,712. This conversion is conducted in a liquid phase reaction, where a Ru containing catalyst and a promoter are dissolved in water, alcohols, ketones or other suitable solvents. Examples of the claimed process are batch processes and methyl acetate is mentioned as by-product.

During methanol synthesis, by-products such as water and small amounts of higher alcohol (C₂-C₅), aldehydes and ketones are formed and the crude methanol is distilled to separate methanol from the by-products. The size and number of distillation columns depend on desired quality of the final methanol product (methanol for fuel purpose or Grade AA methanol).

Consequently, for a given methanol plant the estimation of the exact amount of by-products is important in relation to dimensioning of the actual distillation section. Species like acetone and methyl ethyl ketone with a boiling point close to that of methanol are difficult to remove and consequently the presence of these species will contribute to the demand for a larger and more costly distillation column.

It is thus a general object of the invention to provide an improved process for the production of methanol by catalytic conversion of H₂, CO and CO₂, wherein the produced methanol has a substantially reduced content of aldehyde and ketone impurities.

### SUMMARY OF THE INVENTION

The invention provides a process for production of methanol from a feed stream rich in hydrogen, carbon monoxide and carbon dioxide.

The feed stream is converted to a converted process stream comprising methanol, and small amounts of higher alcohols, aldehydes and ketones in the presence of a catalyst active in conversion of hydrogen and carbon monoxide into methanol, and the converted process stream is cooled to a cooled process stream to 20-200° C.

The cooled process stream is hydrogenated into a hydrogenated process stream rich in methanol and depleted in aldehydes and ketones in presence of a hydrogenation catalyst active in conversion of aldehydes and ketones into alcohols in presence of methanol.

The hydrogenated process stream is cooled and subsequently condensed, and the thus treated process stream is separated into a gas phase and a liquid crude methanol phase.

The hydrogenation can be performed in a reactor or conversion to methanol and hydrogenation may be carried out in the same reactor. Optionally, the hydrogenation is performed in a tubular reactor being cooled by the feed stream to the methanol conversion or in any other way being integrated into the main process.

The hydrogenation of the cooled process gas in presence of the catalyst considerably decreases the content of aldehydes and ketones in the effluent from the synthesis. By the above process a notable fraction of the most difficult by-products, acetone and methyl-ethyl ketone is hydrogenated into the corresponding alcohols, 2-propanol and 2-buthanol, and the down stream distillation for obtaining chemical grade methanol is much simpler.

Removal of methyl-ethyl ketone and acetone to the level requested for Federal Grade AA methanol usually requires a distillation system, which by the above invention will be more simple.

### BRIEF DESCRIPTION OF THE DRAWINGS

FIG. 1 is a graph showing the relation between temperature and theoretical equilibrium amount of acetone and methyl-ethyl ketone.

FIG. 2 is a schematic presentation of the invention.

FIG. 3 is a sectional view of a reactor according to one embodiment of the invention.

### DETAILED DESCRIPTION OF THE INVENTION

The invention is based on hydrogenation of the gas leaving the methanol synthesis reactor (catalyst) at temperatures lower than the exit temperature of the gas leaving the methanol converter (catalyst). The purpose of the hydrogenation step is to lower the amounts of aldehyde and ketone by-products by hydrogenation of the aldehydes and ketones into the corresponding alcohols.

On a Cu-based catalyst, methanol is produced from synthesis gas via the following reactions
CO₂+3H₂═CH₃OH+H₂O (1)
CO+H₂O═CO₂+H₂ (2)

By-products such as higher alcohols may be formed via
nCO+2nH₂═CnH₂ₙ₊₁OH+(n−1)H₂O (3).

Experiments in the methanol test unit in our laboratory as well as analysis of raw methanol from the methanol industry show that acetone and methyl-ethyl-ketone are present in the product stream. Compared to the concentration of ketones only minor amounts of aldehydes are present.

Production of chemical grade methanol requires an extensive purification of the raw methanol by which water and by-products are removed so the specification for e.g. Federal grade AA methanol is satisfied. The most difficult species to remove by distillation is the ones with boiling points close to methanol, see Table 1.

TABLE 1

| | | Boiling Point |
|---|---|---|
| | Boiling Point | Hydrogenation |
| Compound | Feed | Effluent |
| Methanol | 64.7 | |
| Ethanol | 78.4 | |
| Acetone | 56.5 | |
| Methyl-ethyl ketone | 79.6 | |
| iso-propanol | | 82.5 |
| iso-butanol | | 99.5 |

The oxygenate by-products such as ethanol, acetone and methyl-ethyl ketone etc are formed in small quantities during methanol synthesis. The rate of their formation increases with temperature, but also with the CO content of the methanol synthesis gas.

It has now been found that hydrogenation of these ketones are possible on a Cu-based methanol synthesis catalyst and follows the reactions:
CH₃COCH₃+H₂═CH₃—CHOH—CH₃ (4)
CH₃—CH₂—CO—CH₃+H₂═CH₃—CH₂—CHOH—CH₃ (5)

Reactions (4) and (5) are exothermic which implies that the equilibrium between the ketone and the corresponding alcohol is favoured towards that of alcohol at lower temperature.

The experiments indicate further that a Cu-based catalyst is active in hydrogenation of ketones down to a temperature around 150° C.

The exit temperature from an industrial methanol catalyst is typically around 240-260° C. If the ketones in the process gas are equilibrated with respect to the corresponding alcohols at for example 180° C., then the amount of ketones will be lowered by a factor between 6-12 (depending on exit temperatures of the methanol synthesis catalyst).

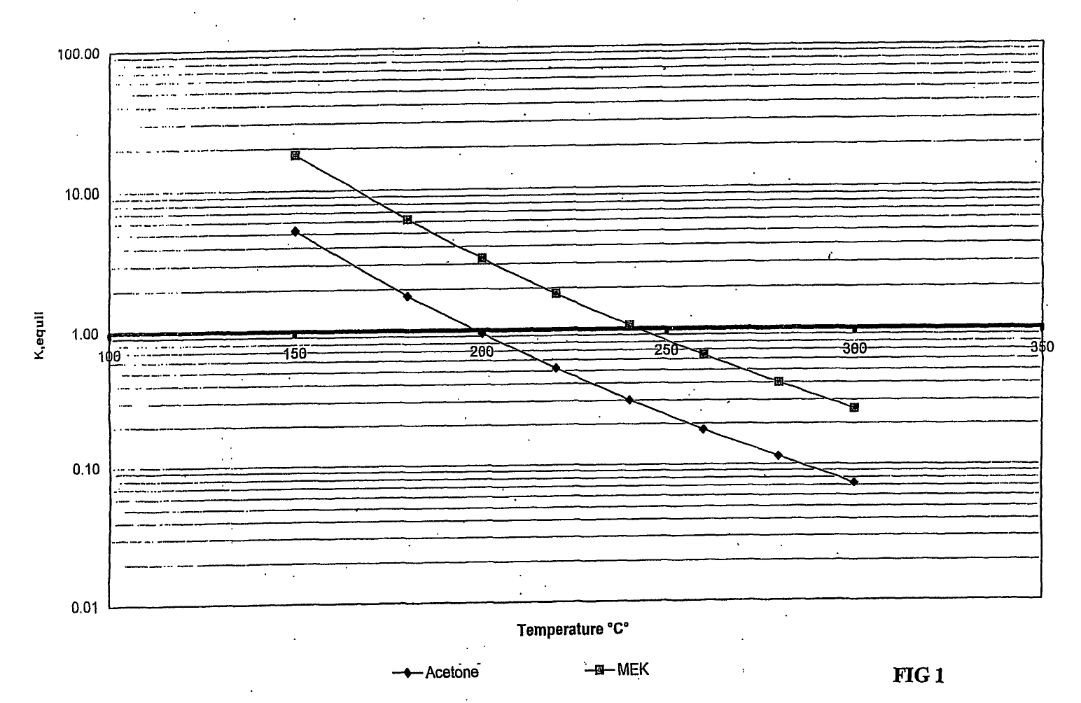

Further, equilibration at say 100° C. will reduce the ketone content with at least a factor of 100. This is seen from the curve on FIG. 1.

In one embodiment of the invention, a ketone hydrogenation converter is arranged after the methanol synthesis converter.

In another embodiment of the invention, the ketone hydrogenation converter is installed as a “feed-effluent” heat exchanger, which means that the exit gas from the synthesis is cooled by heat exchange with fresh synthesis gas to the methanol synthesis.

The catalyst can be in form of pellets, extrudates or powder. And as the hydrogenation activity of the Cu-based catalysts is very high, the catalyst for hydrogenation may be present in a monolithic form or as catalyzed hardware, the benefit is low pressure-drop.

The ketone hydrogenation can furthermore be carried out after condensation of methanol using known hydrogenation catalysts, such as base metal (Cu, Ni) or noble metal based catalysts.

The hydrogenation can take place as an integrated part of the synthesis reactor e.g. the synthesis reactor is operated at low exit temperature (150-200° C.).

A suitable hydrogenation catalyst is a Cu based catalyst with 10-95 wt % Cu, most often 40-70%.

As long as the hydrogenation is carried out in methanol synthesis gas, Cu-based catalysts are preferred, since the Ni-based as well as the noble metal based catalysts may at higher temperature catalyse parasitic reactions like methane formation.

Particular suitable catalysts for the hydrogenation contain noble metal including Pt and Pd. Base metal catalysts like a 10 wt % Ni—Cu catalyst have been mentioned in the art. U.S. Pat. No. 5,243,095 claim a Cu, Fe, Mn, Al based catalyst for ketone hydrogenation and U.S. Pat. No. 3,925,490 claim a Cu, Cr catalyst.

In a preferred embodiment a high activity methanol catalyst can be used as hydrogenation catalyst. A further advantage is that the methanol synthesis can be further completed in a cooled reactor with hydrogenation of the by-products as well.

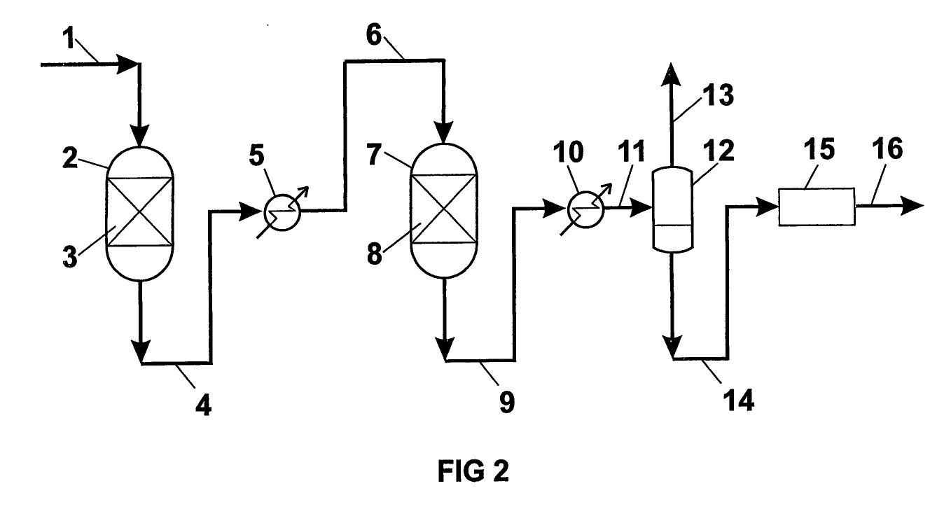

The process is illustrated on FIG. 2, where feed stream 1 enters methanol converter 2. The feed stream comprises hydrogen, carbon monoxide and carbon dioxide, which are converted to mainly methanol and to small amounts of higher alcohols, aldehydes and ketones. The conversion takes place over a catalyst 3 loaded in converter 2. The catalyst is a conventional methanol synthesis catalyst. The converted process stream 4 is cooled in cooler 5 to 200° C., preferably to 150° C., and the cooled process stream 6 flows to hydrogenator 7, which is loaded with hydrogenation catalyst 8. The catalyst is active in hydrogenating aldehydes and ketones to methanol and higher alcohols in a process stream rich in methanol, where also CO is present. The hydrogenated process stream 9 is transferred to a cooler 10, possibly a water cooler, where stream 9 is cooled and condensed together with components with a higher dew point. The cooled, condensed process stream 11 is sent to phase separator 12, where gas phase 13 is withdrawn, possibly returned to 2. Liquid phase, crude methanol 14, is withdrawn and sent to distillation unit 15. In unit 15 the crude methanol is purified to chemical grade methanol 16.

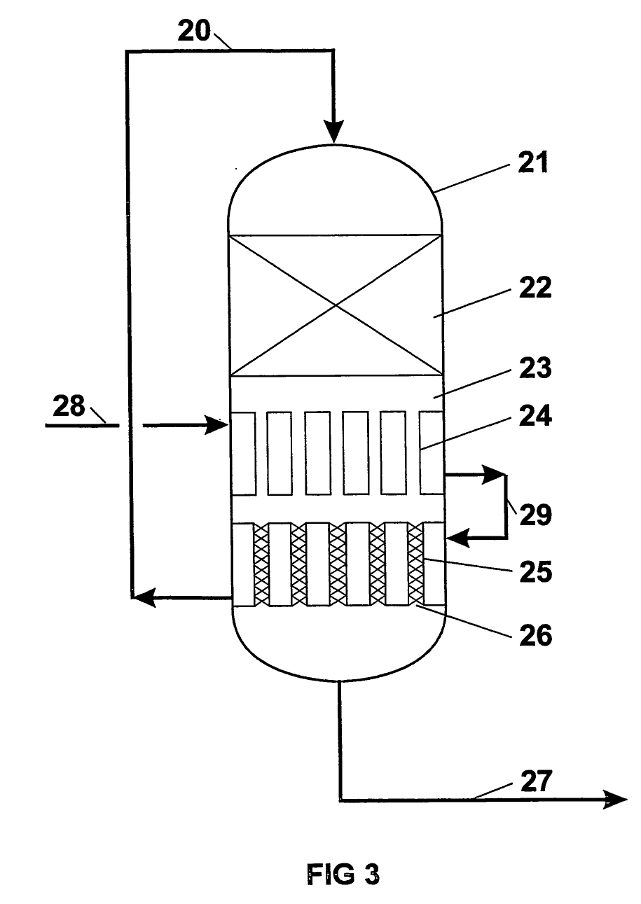

One embodiment of a reactor according to the invention is shown on FIG. 3. Feed gas 20 is introduced to reactor 21, where it passes catalyst 22. Catalyst 22 promotes the conversion of hydrogen, carbon monoxide and carbon dioxide to methanol and by-products as aldehydes, ketones and higher alcohols. The converted process gas 23 flows through an internal cooler 24 and to a tubular hydrogenator 25. The hydrogenator comprises a number of tubes, which are either filled with catalyst pellets or internally coated with hydrogenation catalyst 26. Unconverted gas and crude methanol 27 leave the bottom of reactor 21. Fresh feed gas 28 is introduced to shell side of cooler 24, where it cools the converted process gas to the appropriate temperature for the hydrogenation reaction. Partly preheated fresh gas 29 enters shell side of the tubular hydrogenator 25, where it maintains the reaction temperature and is further preheated before entering reactor 21.

### EXAMPLE 1

Acetone and methyl-ethyl ketone (MEK) are reacted in the presence of a catalyst to form propanol and butanol according to the reaction scheme:
CH₃COCH₃+H₂═CH₃CHOHCH₃
CH₃COC₂H₅+H₂═CH₃CHOHC₂H₅

A Standard Methanol Test Unit has been used. Synthesis gas and different amounts of ketone are fed to the reactor in order to study the ketone hydrogenation activity at various partial pressures. The reactor effluent is cooled, condensed, separated and the liquid phase is depressurised.

The liquid phase is analysed for ketones and alcohols by use of a gas chromatograph.

The feed gas contains, by volume, 5% CO, 5% CO₂, 3% Ar and H₂ as balance. Inlet concentration of ketones is varied between 0.7 and 90 ppm. Reaction pressure is 68 Bar g, the temperature is varied from 150° C. to 240° C. and space velocity is in the range of 10000-60000 Nl/kg/hr.

The reaction takes place over a hydrogenation catalyst available from Haldor Topsøe A/S, Denmark. It contains 45% Cu, 20% Zn and 4% Al by weight.

The measured conversion of acetone and methyl-ethyl ketone at temperatures between 180-240° C. is shown in Table 2. The measured conversion of ketones are close to the theoretical maximum values calculated from the known values of the equilibrium constant and the value of the hydrogen partial pressure at reactor exit conditions and shown in the last column of Table 2. The accuracy of the measurements is around 1% on the shown conversion, which explains the few experimental results higher than the corresponding theoretic figure.

However, the results shown in Table 2 clearly verify that the Cu, Zn, Al catalyst is active in hydrogenation of acetone and methyl-ethyl ketone at temperatures down to 180° C.

TABLE 2
CATALYST:
Cu, Zn, Al
Acetone

| | Partial | | | | Conversion of | |
|---|---|---|---|---|---|---|
| | Pressure | | Condensate | | Acetone | |
| | Inlet, | Exit, | Analysis | | | Equil. |
| T | Acetone | H2 | 2-PrOH | Acetone | Measured | Value |
| [° C.] | [Bar] | [Bar] | [ppm] | [ppm] | [%] | [%] |
| 240 | 0.0028 | 57.0 | 1341 | 26 | 98.1 | 94.5 |
| 220 | 0.0034 | 57.4 | 1906 | 20 | 99.0 | 96.8 |
| 200 | 0.0035 | 58.7 | 3967 | 18 | 99.5 | 98.2 |
| 180 | 4.6E−05 | 59.8 | 188 | 3 | 98.4 | 99.1 |

MEK

| | Partial | | | | Conversion | |
|---|---|---|---|---|---|---|
| | Pressure | | Condensate | | of MEK | |
| | Inlet, | Exit, | Analysis | | | Equil. |
| T | MEK | H2 | 2-BuOH | MEK | Measured | Value |
| [° C.] | [Bar] | [Bar] | [ppm] | [ppm] | [%] | [%] |
| 240 | 3.3E−03 | 57 | 1126 | 53 | 95.5 | 98.4 |
| 220 | 3.5E−03 | 57.8 | 1763 | 44 | 97.6 | 99.1 |
| 200 | 2.1E−03 | 58.8 | 4061 | 46 | 98.9 | 99.5 |
| 180 | 1.6E−03 | 59.3 | 16962 | 62 | 99.6 | 99.7 |
| 180 | 4.2E−03 | 59.4 | 24001 | 75 | 99.7 | 99.7 |
| 180 | 6.3E−03 | 59.7 | 35744 | 105 | 99.7 | 99.7 |
| 180 | 4.6E−05 | 59.8 | 608 | 7 | 98.9 | 99.7 |

### EXAMPLE 2

The experiment of Example 1 was repeated, however, with a different catalyst containing 35% Cu and 28% Al by weight commercially available from Haldor Topsøe A/S, Denmark.

The measured conversion of acetone and methyl-ethyl ketone (MEK) at temperatures between 150° C. and 220° C. is shown in Table 3. The measured conversion of ketones are close to the theoretical maximum values calculated from the known values of equilibrium constant and the value of the hydrogen partial pressure at reactor exit conditions.

The results shown in Table 3 verify that the Cu, Al catalyst is active in hydrogenation of acetone and methyl-ethyl ketone at temperatures down to 150° C.

TABLE 3
CATALYST
Cu, Al
Acetone

| | Partial | | | | Conversion of | |
|---|---|---|---|---|---|---|
| | Pressure | | Condensate | | Acetone | |
| | Inlet, | Exit, | Analysis | | | Equil. |
| T | Acetone | H2 | 2-PrOH | Acetone | Measured | Value |
| [° C.] | [Bar] | [Bar] | [ppm] | [ppm] | [%] | [%] |
| 220 | 5.00E−05 | 60.3 | 258 | 6 | 97.7 | 96.9 |
| 200 | 5.00E−05 | 60.3 | 528 | 5 | 99.1 | 98.3 |
| 150 | 5.0E−05 | 60.4 | 704 | 5 | 99.3 | 99.7 |

MEK

| | Partial | | | | Conversion | |
|---|---|---|---|---|---|---|
| | Pressure | | Condensate | | of MEK | |
| | Inlet, | Exit, | Analysis | | | Equil. |
| T | MEK | H2 | 2-BuOH | MEK | Measured | Value |
| [° C.] | [Bar] | [Bar] | [ppm] | [ppm] | [%] | [%] |
| 220 | 5.0E−05 | 60.3 | 289 | 8 | 97.3 | 99.1 |
| 200 | 5.0E−05 | 60.3 | 565 | 5 | 99.1 | 99.5 |
| 150 | 5.0E−05 | 60.4 | 795 | 2 | 99.7 | 99.9 |

Lately very large capacity plants are being planned and in these situations production of synthesis gas by means of autothermal reforming have become attractive. The resulting synthesis gas composition if made in the most energy efficient manner has a large content of Carbon Monoxide and the formation of by-products during methanol synthesis will increase dramatically.

Note that application of the above technology not only allows a more efficient and cheaper separation sequence, but also opens up for operation of the synthesis reactors at conditions previously not used due to the high by-product content.

1. A process for production of methanol from a feed stream rich in hydrogen, carbon monoxide and carbon dioxide, comprising the steps of
(a) conversion of the feed stream into a converted process stream comprising methanol, aldehydes and ketones in the presence of a catalyst active in conversion of hydrogen, carbon monoxide and carbon dioxide into methanol;
(b) first cooling of the converted process stream to a cooled process stream to 20-200° C.;
(c) hydrogenation of the cooled process stream into a hydrogenated process stream rich in methanol and depleted in aldehydes and ketones in presence of a hydrogenation catalyst active in conversion of aldehydes and ketones into alcohols;
(d) second cooling of the hydrogenated process stream to a cooled, condensed process stream; and
(e) phase separation of the cooled, condensed process stream into a gas phase and a liquid crude methanol.

2. A process according to claim 1, wherein the converted process stream is cooled to 80-150° C.

3. A process according to claim 1, wherein the hydrogenation takes place in a separate reactor.

4. A process according to claim 1, wherein the conversion and the hydrogenation take place in a single reactor.

5. A process according to claim 1, wherein the hydrogenation takes place in a tubular reactor being cooled by a cold feed stream to the conversion.

6. A process according to claim 1, wherein the hydrogenation catalyst is a Cu based catalyst.

7. A process according to claim 6, wherein the Cu content of the hydrogenation catalyst is in the range of 10-95% by weight, preferably 40-70% by weight.

8. A process according to claim 1, wherein the hydrogenation catalyst is a noble metal based catalyst.

9. A process according to claim 1, wherein the hydrogenation catalyst is in the form of pellets, extrudates, monolith, catalysed hardware or a powder suspended in a liquid methanol phase.
